(19) United States
(12) Patent Application Publication
(10) Pub. No.: US 2014/0378922 A1
(43) Pub. Date: Dec. 25, 2014

(54) FLUID-ABSORBENT ARTICLE WITH INDICATOR

(71) Applicant: BASF SE, Ludwigshafen (DE)

(72) Inventors: Andre Fuchs, Schliengen-Obereggenen (DE); Marina Schiller, Rheinfelden (DE)

(73) Assignee: BASF SE, Ludwigshafen (DE)

(21) Appl. No.: 14/311,680

(22) Filed: Jun. 23, 2014

(30) Foreign Application Priority Data
Jun. 25, 2013 (EP) 13173571.4

(51) Int. Cl. A61F 13/42; A61F 13/537

(52) U.S. Cl. CPC A61F 13/42; A61F 13/53713; A61F 2013/422
USPC 604/361; 436/86

(57) ABSTRACT

The present invention relates to a fluid-absorbent article, comprising an indicator for feces especially of newborn and breast-feeded babies, which is applied to a carrier material and to an indicator for feces especially of newborn and breast-feeded babies and substances present in urine indicating kidney or vascular diseases, suitable for fluid-absorbent articles such as diapers, comprising at least one indicator-substance which changes color as a result of contact with proteins.

The present invention relates to a fluid-absorbent article, comprising an indicator for feces especially of newborn and breast-feeded babies, which is applied to a carrier material and to an indicator for feces especially of newborn and breast-feeded babies and substances present in urine indicating kidney or vascular diseases, suitable for fluid-absorbent articles such as diapers, comprising at least one indicator-substance which changes color as a result of contact with proteins.

The production of fluid-absorbent articles such as diapers is described in the monograph “Modern Superabsorbent Polymer Technology”, F. L. Buchholz and A. T. Graham, Wiley-VCH, 1998, pages 252 to 258.

Disposable fluid-absorbent articles such as diapers, sanitary napkins, training pants, adult incontinence products are widely used.

Fluid-absorbent articles consist typically of an upper liquid-pervious top-sheet, a lower liquid-impervious layer, an absorption and distribution layer and fluid-absorbent composite (together “absorbent core”) between the top-sheet or upper liquid-pervious layer and the lower liquid-impervious layer. The composite consists of fluid-absorbent polymers and fibers. Further layers are, for example tissue layers.

Fluid-absorbent polymers are known. The preparation of fluid-absorbent polymer particles is likewise described in the monograph “Modern Superabsorbent Polymer Technology”, F. L. Buchholz and A. T. Graham, Wiley-VCH, 1998, pages 71 to 103. The fluid-absorbent polymer particles are also referred to as superabsorbents.

Superabsorbents are materials that are able to take up and retain many times their weight in water, possibly up to several hundred times their weight, even under moderate pressure. Absorbing capacity is usually lower for salt-containing solutions compared to distilled or otherwise de-ionised water. Typically, a superabsorbent has a centrifugal retention capacity (“CRC”, method of measurement see hereinbelow) of at least 5 g/g, preferably at least 10 g/g and more preferably at least 20 g/g. Such materials are also commonly known by designations such as “high-swellability polymer”, “hydrogel” (often even used for the dry form), “hydrogel-forming polymer”, “water-absorbent polymer”, “absorbent gel-forming material”, “swellable resin”, “water-absorbent resin” or the like. The materials in question are crosslinked hydrophilic polymers, in particular polymers formed from (co)polymerised hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products that are swellable in aqueous fluids, examples being guar derivatives, of which water-absorbent polymers based on partially neutralised acrylic acid are most widely used. Superabsorbents are usually produced, stored, transported and processed in the form of dry powders of polymer particles, “dry” usually meaning less than 5 wt.-% moisture content (method of measurement see hereinbelow), although forms in which superabsorbents particles are bound to a web, typically a nonwoven, are also known for some applications, as are superabsorbent fibres.

A superabsorbent transforms into a gel on taking up a liquid, specifically into a hydrogel when as usual taking up water. By far the most important field of use of superabsorbents is the absorbing of bodily fluids. Superabsorbents are used for example in diapers for infants, incontinence products for adults or feminine hygiene products. Examples of other fields of use are as water-retaining agents in market gardening, as water stores for protection against fire, for liquid absorption in food packaging or, in general, for absorbing moisture. Processes for producing superabsorbents are also known. The acrylate-based superabsorbents which dominate the market are produced by radical polymerisation of acrylic acid in the presence of a crosslinking agent (the “internal crosslinker”), usually in the presence of water, the acrylic acid being neutralised to some degree in a neutralisation step conducted prior to or after polymerisation, or optionally partly prior to and partly after polymerisation, usually by adding an alkali, most often an aqueous sodium hydroxide solution. This yields a polymer gel which is comminuted (depending on the type of reactor used, commination may be conducted concurrently with polymerisation) and dried. Usually, the dried powder thus produced (the “base polymer”) is surface crosslinked (also termed surface “post” crosslinked) by adding further organic or polyvalent crosslinkers to generate a surface layer which is crosslinked to a higher degree than the particle bulk. Most often, aluminium sulphate is being used as polyvalent cationic crosslinker. Applying polyvalent metal cations to superabsorbent particles is sometimes not regarded as surface crosslinking, but termed “surface complexing” or as another form of surface treatment, although it has the same effect of increasing the number of bonds between individual polymer strands at the particle surface and thus increases gel particle stiffness as organic surface crosslinkers have. Organic and polyvalent cation surface crosslinkers can be cumulatively applied, jointly or in any sequence.

Surface crosslinking leads to a higher crosslinking density close to the surface of each superabsorbent particle. This addresses the problem of “gel blocking”, which means that, with earlier types of superabsorbents, a liquid insult will cause swelling of the outermost layer of particles of a bulk of superabsorbent particles into a practically continuous gel layer, which effectively blocks transport of further amounts of liquid (such as a second insult) to unused superabsorbent below the gel layer. While this is a desired effect in some applications of superabsorbents (for example sealing underwater cables), it leads to undesirable effects when occurring in personal hygiene products. Increasing the stiffness of individual gel particles by surface crosslinking leads to open channels between the individual gel particles within the gel layer and thus facilitates liquids transport through the gel layer. Although surface crosslinking decreases the CRC or other parameters describing the total absorption capacity of a superabsorbent sample, it may well increase the amount of liquid that can be absorbed by hygiene product containing a given amount of superabsorbent.

Other means of increasing the permeability of a superabsorbent are also known. These include admixing of superabsorbent with fibres such as fluff in a diaper core or admixing other components that increase gel stiffness or otherwise create open channels for liquid transportation in a gel layer.

In general fluid-absorbent articles are constructed to absorb and hold body waste like urine or feces for extended time periods. But body waste may irritate the skin of the wearer. The skin can become inflamed and irritated. Therefore many different devices comprising sensors to detect wetness or volatile organic compounds have been developed to assist e.g. parents or caregivers identify body waste early on.

WO 2005/067840 for example deals with absorbent articles including an electroactive display used e.g. as wetness indicator

WO 2005/030084 discloses absorbent articles with wetness indicator graphics positioned thereon.

WO 01/95845 also describes absorbent articles, such as diapers, with a wetness indicator applied visible through the backsheet material.

EP 1 216 675 discloses an indicator means for indicating the presence of feces by reacting to the presence of gases given off by faecal matters.

Furthermore WO 99/23985 discloses fiber optic strands having one end within the absorbent core and the opposite end adjacent to the back sheet of an absorbent article for indicating the presence of feces.

Absorbent articles comprising biosensors detecting target biological analytes in bodily waste are also known. These sensors as e.g. disclosed in U.S. Pat. No. 7,982,088 allow the indication of pathogenic bacteria or viruses in bodily waste.

But most of the devices or sensors are very expensive especially the electronic based devices and therefore from an economic and environmental viewpoint not recommendable for disposable articles.

Furthermore especially the indicators for feces are not suitable for newborn and/or breast-feeded babies, as there are reacting to the presence of gases given by faecal matters. But the feces especially of newborn and breast-feeded babies differ in respect to composition and odor from older children and adults. Especially there is less or even no intensive odor signalizing parents or caretakers the presence of faecal matters.

It is therefore an object of the present invention to provide fluid-absorbent articles with an indicator suitable for detecting feces of newborn and breast-feeded babies.

Furthermore it is also an object of the present invention to provide a cost-efficient indicator suitable for fluid-absorbent articles, for feces especially of newborn and breast-feeded babies and substances present in urine indicating kidney or vascular diseases.

The object is achieved by a fluid-absorbent article, comprising

(A) an upper liquid-pervious layer,

(B) a lower liquid-impervious layer and

(C) a fluid-absorbent core between (A) and (B), comprising 0 to 90% by weight fibrous material and 10 to 100% by weight water-absorbent polymer particles,

(D) optionally an acquisition-distribution layer between (A) and (C), comprising 80 to 100% by weight fibrous material and 0 to 20% by weight water-absorbent polymer particles, and

(E) optionally at least one additional layer disposed immediately above and/or below (C),

an indicator for feces applied to a carrier material,

wherein the carrier material is any one of the upper liquid-pervious layer, the lower liquid-impervious layer, the fluid-absorbent core, the water-absorbent polymer particles and the additional layer.

The object is also achieved by a fluid-absorbent article, comprising

(A) an upper liquid-pervious layer,

(B) a lower liquid-impervious layer and

(C) a fluid-absorbent core between (A) and (B), comprising 0 to 90% by weight fibrous material and 10 to 100% by weight water-absorbent polymer particles,

(D) optionally an acquisition-distribution layer between (A) and (C), comprising 80 to 100% by weight fibrous material and 0 to 20% by weight water-absorbent polymer particles, and

(E) optionally at least one additional layer disposed immediately above and/or below (C),

an indicator for feces applied to a carrier material

wherein the carrier material is any one of a membrane or a layer fastened on the upper liquid pervious layer, the lower liquid-impervious layer, the fluid-absorbent core or the additional layer.

Furthermore the object is achieved by an indicator for feces especially of newborn and breast-feeded babies and substances present in urine indicating kidney or vascular diseases, suitable for fluid-absorbent articles such as diapers, comprising at least one indicator-substance which changes color as a result of contact with proteins, whereas the indicator is applied on a carrier material.

According to the invention the indicator comprises at least one indicator-substance which changes color as a result of contact with proteins.

The reaction of the indicator with the feces respectively cause a visually discernable color change of the at least one indicator-substance.

Furthermore the indicator also shows a color change in reaction with substances present in urine especially of adult people in case of kidney or vascular diseases. Therefore the indicator also indicates kidney or vascular diseases.

A preferred indicator-substance is ninhydrin, which chemically reacts in the presence of aminoacids, amines and amino sugars to form a vivid purple product. Ninhydrin can detect a protein by reacting to the amino group of the protein.

A carrier material according to the invention is a supporting structure for carrying the indicator. Useful carrier materials are disclosed in WO2009/005884, such as fluff, flexible ceramic sheets, films, woven or knitted materials, nonwovens, paper, tissue, foams, sponges or membranes or a variety of combinations thereof. Preferred carrier material is white or transparent and the indicator has a good adhesion to the surface of the material. In a preferred embodiment the material is porous.

According to the invention the indicator may be applied on a carrier material, which may be the inside, the side adjacent to the absorbent core, of the liquid impervious layer or any one of the upper liquid-pervious layer, the lower liquid-impervious layer, the fluid-absorbent core and the additional layer or a carrier material, e.g. in form of a layer or strip or membrane fastened on the inside of the liquid impervious layer.

Furthermore fluid-absorbent polymer particles may also serve as carrier material.

Wherein it is preferred that the indicator is applied in such a way, that the color change is visible through the material of e.g. the liquid impervious layer of the fluid absorbent article.

Therefore it is preferred that the indicator is applied on or adjacent to at least one part of the liquid impervious layer having a color or transparency allowing the indicator to be visible through the impervious layer material.

In one embodiment the indicator may be dispersed within the water-absorbent polymer, wherein the fluid-absorbent polymer may be placed in discrete regions of the absorbent core, whereas at least one part of the liquid impervious layer and/or the absorbent core having a color or transparency allowing the indicator to be visible through the impervious layer material.

Suitable fluid-absorbent polymer particles for the inventive fluid-absorbent articles are described in e.g. EP 1 770 113, WO 04/113452, WO 00/053644, WO 00/053664, WO 02/20068, WO 02/22717, WO 06/42704, WO 08/9580.

The fluid absorbent core (C) of the fluid absorbent-article comprises typically at least 60%, preferably at least 70%, more preferred at least 80% and most preferred at least 90% by weight of fluid-absorbent polymer particles.

The fluid absorbent core (C) of the fluid absorbent article may contain different amounts of fluid-absorbent polymer particles depending on targeted use. For example a maxi size/L/04 diaper contains at least 8 g, more preferably at least 11 g, most preferably at least 13 g of the fluid-absorbent polymer particles.

Suitable fluid-absorbent polymer particles for the inventive fluid-absorbent articles have a saline flow conductivity (SFC) of at least 8×10⁻⁷ cm³ s/g, typically at least 20×10⁻⁷ cm³ s/g, preferably at least 25×10⁻⁷ cm³ s/g, preferentially preferably at least 30×10⁻⁷ cm³ s/g, most preferably at least 50×10⁻⁷ cm³ s/g. The saline flow conductivity (SFC) of the fluid-absorbent polymer particles is typically less than 500×10⁻⁷ cm³ s/g.

Suitable fluid-absorbent polymer particles for the fluid-absorbent articles according to the invention have a centrifuge retention capacity (CRC) preferably of at least 20 g/g, most preferably of at least 24 g/g. The centrifuge retention capacity (CRC) of the fluid-absorbent polymer particles is typically less than 60 g/g.

Suitable fluid-absorbent polymer particles for the inventive fluid-absorbent articles have a absorbency under high load of typically at least 18 g/g, preferably at least 20 g/g, more preferably at least 22 g/g, most preferably at least 24 g/g. The absorbency under high load of the fluid-absorbent polymer particles is typically less than 35 g/g.

### DETAILED DESCRIPTION OF THE INVENTION

### A. Definitions

As used herein, the term “fluid-absorbent article” refers to any three-dimensional solid material being able to acquire and store fluids discharged from the body. Preferred fluid-absorbent articles are disposable fluid-absorbent articles that are designed to be worn in contact with the body of a user such as disposable fluid-absorbent pantyliners, sanitary napkins, catamenials, incontinence inserts/pads, diapers, training pant diapers, breast pads, interlabial inserts/pads or other articles useful for absorbing body fluids.

As used herein, the term “fluid-absorbent composition” refers to a component of the fluid-absorbent article which is primarily responsible for the fluid handling of the fluid-absorbent article including acquisition, transport, distribution and storage of body fluids.

As used herein, the term “fluid-absorbent core” refers to a fluid-absorbent composition comprising fluid-absorbent polymer particles and a fibrous material. The fluid-absorbent core is primarily responsible for the fluid handling of the fluid-absorbent article including acquisition, transport, distribution and storage of body fluids.

As used herein, the term “layer” refers to a fluid-absorbent composition whose primary dimension is along its length and width. It should be known that the term “layer” is not necessarily limited to single layers or sheets of the fluid-absorbent composition. Thus a layer can comprise laminates, composites, combinations of several sheets or webs of different materials.

As used herein the term “x-dimension” refers to the length, and the term “y-dimension” refers to the width of the fluid-absorbent composition, layer, core or article. Generally, the term “x-y-dimension” refers to the plane, orthogonal to the height or thickness of the fluid-absorbent composition, layer, core or article.

As used herein the term “z-dimension” refers to the dimension orthogonal to the length and width of the fluid absorbent composition, layer, core or article. Generally, the term “z-dimension” refers to the height of the fluid-absorbent composition, layer, core or article.

As used herein, the term “density” indicates the weight of the fluid-absorbent core per volume and it includes the chassis of the fluid-absorbent article. The density is determined at discrete regions of the fluid-absorbent core: the front overall average is the density of the fluid-absorbent core 5.5 cm forward of the center of the core to the front distal edge of the core; the insult zone is the density of the fluid-absorbent core 5.5 cm forward and 0.5 cm backwards of the center of the core; the back overall average is the density of the fluid-absorbent core 0.5 cm backward of the center of the core to the rear distal edge of the core.

Further, it should be understood, that the term “upper” refers to fluid-absorbent composition which are nearer to the wearer of the fluid-absorbent article. Generally, the topsheet is the nearest composition to the wearer of the fluid-absorbent article, hereinafter described as “upper liquid-pervious layer”. Contrarily, the term “lower” refers to fluid-absorbent compositions which are away from the wearer of the fluid-absorbent article. Generally, the backsheet is the component which is furthermost away from the wearer of the fluid-absorbent article, hereinafter described as “lower liquid-impervious layer”.

As used herein, the term “liquid-pervious” refers to a substrate, layer or a laminate thus permitting liquids, i.e. body fluids such as urine, menses and/or vaginal fluids to readily penetrate through its thickness.

As used herein, the term “liquid-impervious” refers to a substrate, layer or a laminate that does not allow body fluids to pass through in a direction generally perpendicular to the plane of the layer at the point of liquid contact under ordinary use conditions.

As used herein, the term “chassis” refers to fluid-absorbent material comprising the upper liquid-pervious layer and the lower liquid-impervious layer, elastication and closure systems for the absorbent article.

As used herein, the term “hydrophilic” refers to the wettability of fibers by water deposited on these fibers. The term “hydrophilic” is defined by the contact angle and surface tension of the body fluids. According to the definition of Robert F. Gould in the 1964 American Chemical Society publication “Contact angle, wettability and adhesion”, a fiber is referred to as hydrophilic, when the contact angle between the liquid and the fiber, especially the fiber surface, is less than 90° or when the liquid tends to spread spontaneously on the same surface.

Contrarily, term “hydrophobic” refers to fibers showing a contact angle of greater than 90° or no spontaneously spreading of the liquid across the surface of the fiber.

As used herein, the term “body fluids” refers to any fluid produced and discharged by human or animal body, such as urine, menstrual fluids, faeces, vaginal secretions and the like.

As used herein, the term “breathable” refers to a substrate, layer, film or a laminate that allows vapour to escape from the fluid-absorbent article, while still preventing fluids from leakage. Breathable substrates, layers, films or laminates may be porous polymeric films, nonwoven laminates from spunbond and melt-blown layers, laminates from porous polymeric films and nonwovens.

As used herein, the term “longitudinal” refers to a direction running perpendicular from a waist edge to an opposing waist edge of the fluid-absorbent article.

### B. Fluid-absorbent Polymer Particles

The production of fluid-absorbent polymer particles is described in the monograph “Modern Superabsorbent Polymer Technology”, F. L. Buchholz and A. T. Graham, Wiley-VCH, 1998, pages 71 to 103.

The preparation of spherical fluid-absorbent polymer particles by polymerizing droplets of a monomer solution is described, for example, in EP 0 348 180 A1, WO 96/40427 A1, U.S. Pat. No. 5,269,980, DE 103 14 466 A1, DE 103 40 253 A1, DE 10 2004 024 437 A1, DE 10 2005 002 412 A1, DE 10 2006 001 596 A1, WO 2008/009580 A1, WO 2008/009598 A1, WO 2008/009599 A1 and WO 2008/009612 A1, and also PCT/EP2008/051336 and PCT/EP2008/051353.

Polymerization of monomer solution droplets in a gas phase surrounding the droplets (“dropletization polymerization” affords round fluid-absorbent polymer particles of high mean sphericity (mSPHT). The mean sphericity is a measure of the roundness of the polymer particles and can be determined, for example, with the Camsizer® image analysis system (Retsch Technology GmbH, Haan, Germany). The water-absorbent poly particles obtained by dropletization polymerization are typically spheres with one or more cavities. The water-absorbent polymers can be divided into three categories: water-absorbent polymer particles of Type 1 are particles with one cavity, water absorbent polymer particles of Type 2 are particles with more than one cavity, and water-absorbent polymer particles of Type 3 are solid particles with no visible cavity.

The morphology of the fluid-absorbent polymer particles can be controlled by the reaction conditions during polymerization. Fluid-absorbent polymer particles having a high amount of particles with one cavity (Type 1) can be prepared by using low gas velocities and high gas exit temperatures. Fluid-absorbent polymer particles having a high amount of particles with more than one cavity (Type 2) can be prepared by using high gas velocities and low gas exit temperatures.

Fluid-absorbent polymer particles having more than one cavity (Type 2) show an improved mechanical stability.

The fluid-absorbent polymer particles are produced, for example, by polymerizing a monomer solution or suspension comprising

a) at least one ethylenically unsaturated monomer which bears acid groups and may be at least partly neutralized,

b) at least one crosslinker,

c) at least one initiator,

d) optionally one or more ethylenically unsaturated monomers copolymerizable with the monomers mentioned under a) and

e) optionally one or more water-soluble polymers,

and are typically water-insoluble.

Suitable monomers a) are, for example, ethylenically unsaturated carboxylic acids such as acrylic acid, methacrylic acid, maleic acid, and itaconic acid. Particularly preferred monomers are acrylic acid and methacrylic acid. Very particular preference is given to acrylic acid.

### B. Fluid-absorbent articles

The fluid-absorbent article comprises

(A) an upper liquid-pervious layer

(B) a lower liquid-impervious layer

(C) a fluid-absorbent core between (A) and (B) comprising

at least 5 to 90% by weight a fibrous material and from 10 to 95% by weight water-absorbent polymer particles;

preferably at least 20 to 80% by weight a fibrous material and from 20 to 80% by weight water-absorbent polymer particles;

more preferably at least 30 to 75% by weight a fibrous material and from 25 to 70% by weight water-absorbent polymer particles;

most preferably at least 40 to 70% by weight a fibrous material and from 30 to 60% by weight water-absorbent polymer particles;

and an optional dusting layer

(D) an optional acquisition-distribution layer between (A) and (C), comprising

80 to 100% by weight fibrous material and from 0 to 20% by weight water-absorbent polymer particles

preferably at least 85 to 99.9% by weight a fibrous material and from 0.01 to 15% by weight water-absorbent polymer particles;

more preferably at least 90% to 99.5% by weight a fibrous material and from 0.5 to 10% by weight water-absorbent polymer particles;

most preferably at least 95-99% by weight fibrous material and from 1 to 5% by weight water-absorbent polymer particles;

(E) an optional tissue layer disposed immediately above and/or below (C); and

(F) other optional components.

Fluid-absorbent articles are understood to mean, for example, incontinence pads and incontinence briefs for adults or diapers for babies. Suitable fluid-absorbent articles including fluid-absorbent compositions comprising fibrous material and optionally fluid-absorbent polymer particles to form fibrous webs or matrices for the substrates, layers, sheets and/or the fluid-absorbent core.

Suitable fluid-absorbent articles are composed of several layers whose individual elements must show preferably definite functional parameters such as dryness for the upper liquid-pervious layer, vapor permeability without wetting through for the lower liquid-impervious layer, a flexible, vapor permeable and thin fluid-absorbent core, showing fast absorption rates and being able to retain highest quantities of body fluids, and an acquisition-distribution layer between the upper layer and the core, acting as transport and distribution layer of the discharged body fluids. These individual elements are combined such that the resultant fluid-absorbent article meets overall criteria such as flexibility, water vapour breathability, dryness, wearing comfort and protection on the one side, and concerning liquid retention, rewet and prevention of wetting through on the other side. The specific combination of these layers provides a fluid-absorbent article delivering both high protection levels as well as high comfort to the consumer.

I. Liquid-pervious Layer (A)

The liquid-pervious layer (A) is the layer which is in direct contact with the skin. Thus, the liquid-pervious layer is preferably compliant, soft feeling and non-irritating to the consumer's skin. Generally, the term “liquid-pervious” is understood thus permitting liquids, i.e. body fluids such as urine, menses and/or vaginal fluids to readily penetrate through its thickness. The principle function of the liquid-pervious layer is the acquisition and transport of body fluids from the wearer towards the fluid-absorbent core. Typically liquid-pervious layers are formed from any materials known in the art such as nonwoven material, films or combinations thereof. Suitable liquid-pervious layers (A) consist of customary synthetic non-cellulose based or semisynthetic fibers or bicomponent fibers or films of polyester, polyolefins, rayon or natural cellulose based fibers or any combinations thereof. In the case of nonwoven materials, the fibers should generally be bound by binders such as polyacrylates. Additionally the liquid-pervious layer may contain elastic compositions thus showing elastic characteristics allowing to be stretched in one or two directions.

Suitable synthetic non-cellulose based fibers are made from polyvinyl chloride, polyvinyl fluoride, polytetrafluorethylene, polyvinylidene chloride, polyacrylics, polyvinylacetate, polyethylvinylacetate, non-soluble or soluble polyvinyl alcohol, polylactic acid, polyolefins such as polyethylene, polypropylene, polyamides, polyesters, polyurethanes, polystyrenes and the like.

A detailed overview of examples of fibers which can be used in the present invention is given by the patent application WO 95/26209 A1, page 28 line 9 to page 36 line 8. Said passage is thus part of this invention.

Examples of cellulose fibers include cellulose fibers which are customarily used in absorption products, such as fluff pulp and cellulose of the cotton type. The materials (soft- or hardwoods), production processes such as chemical pulp, semichemical pulp, chemothermomechanical pulp (CTMP) and bleaching processes are not particularly restricted. For example, natural cellulose fibers such as cotton, flax, silk, wool, jute, ethylcellulose and cellulose acetate are used.

Suitable synthetic fibers are produced from polyvinyl chloride, polyvinyl fluoride, polytetrafluoroethylene, polyvinylidene chloride, polyacrylic compounds such as ORLON®, polyvinyl acetate, polyethyl vinyl acetate, soluble or insoluble polyvinyl alcohol. Examples of synthetic fibers include thermoplastic polyolefin fibers, such as polyethylene fibers (PULPEX®), polypropylene fibers and polyethylene-polypropylene bicomponent fibers, polyester fibers, such as polyethylene terephthalate fibers (DACRON® or KODEL®), copolyesters, polyvinyl acetate, polyethyl vinyl acetate, polyvinyl chloride, polyvinylidene chloride, polyacrylics, polyamides, copolyamides, polystyrene and copolymers of the aforementioned polymers and also bicomponent fibers composed of polyethylene terephthalate-polyethylene-isophthalate copolymer, polyethyl vinyl acetate/polypropylene, polyethylene/polyester, polypropylene/polyester, copolyester/polyester, polyamide fibers (nylon), polyurethane fibers, polystyrene fibers and polyacrylonitrile fibers. Preference is given to polyolefin fibers, polyester fibers and their bicomponent fibers. Preference is further given to thermally adhesive bicomponent fibers composed of polyolefin of the core-sheath type and side-by-side type on account of their excellent dimensional stability following fluid absorption.

The fiber cross section may be round or angular, or else have another shape, for example like that of a butterfly.

The synthetic fibers mentioned are preferably used in combination with thermoplastic fibers. In the course of the heat treatment, the latter migrate to some extent into the matrix of the fiber material present and so constitute bond sites and renewed stiffening elements on cooling. In addition, the addition of thermoplastic fibers means that there is an increase in the present pore dimensions after the heat treatment has taken place. This makes it possible, by continuous metered addition of thermoplastic fibers during the formation of the absorbent layer, to continuously increase the fraction of thermoplastic fibers in the direction of the topsheet, which results in a similarly continuous increase in the pore sizes. Thermoplastic fibers can be formed from a multitude of thermoplastic polymers which have a melting point of less than 190° C., preferably in the range from 75° C. to 175° C. These temperatures are too low for damage to the cellulose fibers to be likely.

Examples for films are apertured formed thermoplastic films, apertured plastic films, hydroformed thermoplastic films, reticulated thermoplastic films, porous foams, reticulated foams, and thermoplastic scrims.

Examples of suitable modified or unmodified natural cellulose based fibers include cotton, bagasse, kemp, flax, silk, wool, wood pulp, chemically modified wood pulp, jute, rayon, ethyl cellulose, and cellulose acetate.

Suitable wood pulp fibers can be obtained by chemical processes such as the Kraft and sulfite processes, as well as from mechanical processes, such as ground wood, refiner mechanical, thermo-mechanical, chemi-mechanical and chemi-thermo-mechanical pulp processes. Further, recycled wood pulp fibers, bleached, unbleached, elementally chlorine free (ECF) or total chlorine free (TCF) wood pulp fibers can be used.

The fibrous material may comprise only natural cellulose based fibers or synthetic non-cellulose based fibers or any combination thereof. Preferred materials are polyester, rayon and blends thereof, polyethylene, and polypropylene.

The fibrous material as a component of the fluid-absorbent article may be hydrophilic, hydrophobic or can be a combination of both hydrophilic and hydrophobic fibers. The definition of hydrophilic is given in the section “definitions” in the chapter above. The selection of the ratio hydrophilic/hydrophobic and accordingly the amount of hydrophilic and hydrophobic fibers within fluid-absorbent composition will depend upon fluid handling properties and the amount of fluid-absorbent polymer particles of the resulting fluid-absorbent article. Such, the use of hydrophobic fibers is preferred if the fluid-absorbent article is adjacent to the wearer, that is to be used to replace partially or completely the upper liquid-pervious layer, preferably formed from hydrophobic nonwoven materials. Hydrophobic fibers can also be member of the lower breathable, but fluid-impervious layer, acting there as a fluid-impervious barrier.

Examples for hydrophilic fibers are cellulose based fibers, modified cellulose based fibers, rayon, polyester fibers such as polyethylene terephthalate, hydrophilic nylon and the like. Hydrophilic fibers can also be obtained from hydrophobic fibers which are hydrophilized by e.g. surfactant-treating or silica-treating. Thus, hydrophilic thermoplastic fibers derived from polyolefins such as polypropylene, polyamides, polystyrenes or the like by surfactant-treating or silica-treating.

To increase the strength and the integrity of the upper layer, the fibers should generally show bonding sites, which act as crosslinks between the fibers within the layer.

Technologies for consolidating fibers in a web are mechanical bonding, thermal bonding and chemical bonding. In the process of mechanical bonding the fibers are entangled mechanically, e.g., by water jets (spunlace) to give integrity to the web. Thermal bonding is carried out by means of rising the temperature in the presence of low-melting polymers. Examples for thermal bonding processes are spun-bonding, through-air bonding and resin bonding.

Preferred means of increasing the integrity are thermal bonding, spun-bonding, resin bonding, through-air bonding and/or spunlace.

In the case of thermal bonding, thermoplastic material is added to the fibers. Upon thermal treatment at least a portion of this thermoplastic material is melting and migrates to intersections of the fibers caused by capillary effects. These intersections solidify to bond sites after cooling and increase the integrity of the fibrous matrix. Moreover, in the case of chemically stiffened cellulose based fibers, melting and migration of the thermoplastic material has the effect of increasing the pore size of the resultant fibrous layer while maintaining its density and basis weight. Upon wetting, the structure and integrity of the layer remains stable. In summary, the addition of thermoplastic material leads to improved fluid permeability of discharged body fluids and thus to improved acquisition properties.

Suitable thermoplastic materials including polyolefins such as polyethylene and polypropylene, polyesters, copolyesters, polyvinyl acetate, polyethylvinyl acetate, polyvinyl chloride, polyvinylidene chloride, polyacrylics, polyamides, copolyamides, polystyrenes, polyurethanes and copolymers of any of the mentioned polymers.

Suitable thermoplastic fibers can be made from a single polymer that is a mono-component fiber. Alternatively, they can be made from more than one polymer, e.g., bi-component or multi-component fibers. The term “bi-component fibers” refers to thermoplastic fibers that comprise a core fiber made from a different fiber material than the shell. Typically, both fiber materials have different melting points, wherein generally the sheath melts at lower temperatures. Bi-component fibers can be helical, concentric or eccentric depending whether the sheath has a thickness that is even or uneven through the cross-sectional area of the bi-component fiber. Advantage is given for eccentric bi-component fibers showing a higher compressive strength at lower fiber thickness. Further bi-component fibers can show the feature “uncrimped” (unbent) or “crimped” (bent), further bi-component fibers can demonstrate differing aspects of surface lubricity.

Examples of bi-component fibers include the following polymer combinations: polyethylene/polypropylene, polyethylvinyl acetate/polypropylene, polyethylene/polyester, polypropylene/polyester, copolyester/polyester and the like.

Suitable thermoplastic materials have a melting point of lower temperatures that will damage the fibers of the layer; but not lower than temperatures, where usually the fluid-absorbent articles are stored. Preferably the melting point is between about 75° C. and 175° C. The typical length of thermoplastic fibers is from about 0.4 to 6 cm, preferably from about 0.5 to 1 cm. The diameter of thermoplastic fibers is defined in terms of either denier (grams per 9000 meters) or dtex (grams per 10 000 meters). Typical thermoplastic fibers have a dtex in the range from about 1.2 to 20, preferably from about 1.4 to 10.

A further mean of increasing the integrity of the fluid-absorbent article is the spun-bonding technology. The nature of the production of fibrous layers by means of spunbonding is based on the direct spinning of polymeric granulates into continuous filaments and subsequently manufacturing the fibrous layer.

Spun-bond fabrics are produced by depositing extruded, spun fibers onto a moving belt in a uniform random manner followed by thermal bonding the fibers. The fibers are separated during the web laying process by air jets. Fibre bonds are generated by ap-plying heated rolls or hot needles to partially melt the polymer and fuse the fibers together. Since molecular orientation increases the melting point, fibers that are not highly drawn can be used as thermal binding fibres. Polyethylene or random ethylene/-propylene copolymers are used as low melting bonding sites.

Besides spunbonding, the technology of resin bonding also belongs to thermal bonding subjects. Using this technology to generate bonding sites, specific adhesives, based on e.g. epoxy, polyurethane and acrylic are added to the fibrous material and the resulting matrix is thermally treated. Thus the web is bonded with resin and/or thermal plastic resins dispersed within the fibrous material.

As a further thermal bonding technology through-air bonding involves the application of hot air to the surface of the fibrous fabric. The hot air is circulated just above the fibrous fabric, but does not push through the fibrous fabric. Bonding sites are generated by the addition of binders. Suitable binders used in through-air thermal bonding include crystalline binder fibers, bi-component binder fibers, and powders. When using crystalline binder fibers or powders, the binder melts entirely and forms molten droplets throughout the nonwoven's cross-section. Bonding occurs at these points upon cooling. In the case of sheath/core binder fibers, the sheath is the binder and the core is the carrier fiber. Products manufactured using through-air ovens tend to be bulky, open, soft, strong, extensible, breathable and absorbent. Through-air bonding followed by immediate cold calendering results in a thickness between a hot roll calendered product and one that has been though-air bonded without compression. Even after cold calendering, this product is softer, more flexible and more extensible than area-bond hot-calendered material.

Spunlacing (“hydroentanglement”) is a further method of increasing the integrity of a web. The formed web of loose fibers (usually air-laid or wet-laid) is first compacted and prewetted to eliminate air pockets. The technology of spunlacing uses multiple rows of fine high-speed jets of water to strike the web on a porous belt or moving perforated or patterned screen so that the fibers knot about one another. The water pressure generally increases from the first to the last injectors. Pressures as high as 150 Bar are used to direct the water jets onto the web. This pressure is sufficient for most of the non-woven fibers, although higher pressures are used in specialized applications.

The spunlace process is a nonwovens manufacturing system that employs jets of water to entangle fibers and thereby provide fabric integrity. Softness, drape, conformability, and relatively high strength are the major characteristics of spunlace nonwoven.

In newest researches benefits are found in some structural features of the resulting liquid-pervious layers. For example, the thickness of the layer is very important and influences together with its x-y dimension the acquisition-distribution behaviour of the layer. If there is further some profiled structure integrated, the acquisition-distribution behavior can be directed depending on the three-dimensional structure of the layer. Thus 3D-polyethylene in the function of liquid-pervious layer is preferred.

Thus, suitable liquid-pervious layers (A) are nonwoven layers formed from the fibers above by thermal bonding, spunbonding, resin bonding or through-air bonding. Further suitable liquid-pervious layers are 3D-polyethylene layers and spunlace.

Preferably the 3D-polyethylene layers and spunlace show basis weights from 12 to 22 gsm.

Typically liquid-pervious layers (A) extend partially or wholly across the fluid-absorbent structure and can extend into and/or form part of all the preferred sideflaps, side wrapping elements, wings and ears.

II. Liquid-Impervious Layer (B)

The liquid-impervious layer (B) prevents the exudates absorbed and retained by the fluid-absorbent core from wetting articles which are in contact with the fluid-absorbent article, as for example bedsheets, pants, pyjamas and undergarments. The liquid-impervious layer (B) may thus comprise a woven or a nonwoven material, polymeric films such as thermoplastic film of polyethylene or polypropylene, or composite materials such as film-coated nonwoven material.

Suitable liquid-impervious layers include nonwoven, plastics and/or laminates of plastic and nonwoven. Both, the plastics and/or laminates of plastic and nonwoven may appropriately be breathable, that is, the liquid-impervious layer (B) can permit vapors to escape from the fluid-absorbent material. Thus the liquid-impervious layer has to have a definite water vapor transmission rate and at the same time the level of impermeability. To combine these features, suitable liquid-impervious layers including at least two layers, e.g. laminates from fibrous nonwoven having a specified basis weight and pore size, and a continuous three-dimensional film of e.g. polyvpropylene and/or polyethylene or combinations thereof as the second layer having a specified thickness and optionally having pore structure. Such laminates acting as a barrier and showing no liquid transport or wet through. Thus, suitable liquid-impervious layers comprising at least a first breathable layer of a porous web which is a fibrous nonwoven, e.g. a composite web of a meltblown nonwoven layer or of a spun-bonded nonwoven layer made from synthetic non-cellulose based fibers and at least a second layer of a resilient three dimensional web consisting of a liquid-impervious polymeric film, e.g. plastics optionally having pores acting as capillaries, which are preferably not perpendicular to the plane of the film but are disposed at an angle of less than 90° relative to the plane of the film.

Suitable liquid-impervious layers are permeable for vapor. Preferably the liquid-impervious layer is constructed from vapor permeable material showing a water vapor transmission rate (WVTR) of at least about 100 gsm per 24 hours, preferably at least about 250 gsm per 24 hours and most preferred at least about 500 gsm per 24 hours.

Preferably the liquid-impervious layer (B) is made of nonwoven comprising hydrophobic materials, e.g. synthetic non-cellulose based fibers or a liquid-impervious polymeric film comprising plastics e.g. polyethylene and/or polypropylene and/or combinations thereof. The thickness of the liquid-impervious layer is preferably 12 to 30 μm.

Further, the liquid-impervious layer (B) is preferably made of a laminate of nonwoven and plastics comprising a nonwoven having a density of 12 to 15 gsm and a polyethylene layer having a thickness of about 10 to 20 μm.

The typical liquid-impervious layer (B) extends partially or wholly across the fluid-absorbent structure and can extend into and/or form part of all the preferred sideflaps, side wrapping elements, wings and ears.

III. Fluid-Absorbent Core (C)

The fluid-absorbent core (C) is disposed between the upper liquid-pervious layer (A) and the lower liquid-impervious layer (B). Suitable fluid-absorbent cores (C) may be selected from any of the fluid-absorbent core-systems known in the art provided that requirements such as vapor permeability, flexibility and thickness are met. Suitable fluid-absorbent cores refer to any fluid-absorbent composition whose primary function is to acquire, transport, distribute, absorb, store and retain discharged body fluids.

The top view area of the fluid-absorbent core of a maxi size/L/4 baby diaper (C) is preferably at least 200 cm², more preferably at least 250 cm², most preferably at least 300 cm². The top view area is the part of the core that is face-to-face to the upper liquid-pervious layer.

The fluid absorbent core (C) of the fluid absorbent-article comprises typically at least 60%, preferably at least 70%, more preferred at least 80% and most preferred at least 90% by weight of fluid-absorbent polymer particles.

The fluid absorbent core (C) of the fluid absorbent article may contain different amounts of fluid-absorbent polymer particles depending on targeted use. For example a maxi size/L/04 diaper contains at least 8 g, more preferably at least 11 g, most preferably at least 13 g of the fluid-absorbent polymer particles.

Furthermore the fluid-absorbent core according to the present invention can include the following components:

1. an optional core cover

2. a fluid storage layer

3. an optional dusting layer

### 1. Optional Core Cover

In order to increase the integrity of the fluid-absorbent core, the core is provided with a cover. This cover may be at the top and/or at the bottom of the fluid-absorbent core. Further, this cover may include the whole fluid-absorbent core with a unitary sheet of material and thus function as a wrap. Wrapping is possible as a full wrap, a partial wrap or as a C-Wrap.

The material of the core cover may comprise any known type of substrate, including webs, garments, textiles, films, tissues and laminates of two or more substrates or webs. The core cover material may comprise natural based fibers, such as cellulose, cotton, flax, linen, hemp, wool, silk, fur, hair and naturally occurring mineral fibers. The core cover material may also comprise synthetic fibers such as rayon and lyocell (derived from cellulose), polysaccharides (starch), polyolefin fibers (polypropylene, polyethylene), polyamides, polyester, butadiene-styrene block copolymers, polyurethane and combinations thereof. Preferably, the core cover comprises synthetic non-cellulose based fibers or tissue.

The fibers may be mono- or multicomponent. Multicomponent fibers may comprise a homopolymer, a copolymer or blends thereof.

### 2. Fluid-Storage Layer

The fluid-absorbent compositions included in the fluid-absorbent core comprise fibrous materials and fluid-absorbent polymer particles.

Fibers useful in the present invention include natural cellulose based fibers and synthetic non-cellulose based fibers. Examples of suitable modified or unmodified natural cellulose based fibers are given in the chapter “Liquid-pervious Layer (A)” above. From those, wood pulp fibers are preferred.

Examples of suitable synthetic non-cellulose based fibers are given in the chapter “Liquid-pervious Layer (A)” above.

The fibrous material may comprise only natural cellulose based fibers or synthetic non-cellulose based fibers or any combination thereof.

The fibrous material as a component of the fluid-absorbent compositions may be hydrophilic, hydrophobic or can be a combination of both hydrophilic and hydrophobic fibers.

Generally for the use in a fluid-absorbent core, which is the embedded between the upper layer (A) and the lower layer (B), hydrophilic fibers are preferred. This is especially the case for fluid-absorbent compositions that are desired to quickly acquire, transfer and distribute discharged body fluids to other regions of the fluid-absorbent composition or fluid-absorbent core. The use of hydrophilic fibers is especially preferred for fluid-absorbent compositions comprising fluid-absorbent polymer particles.

Examples for hydrophilic fibers are given in the chapter “Liquid-pervious Layer (A)” above. Preferably, the fluid-absorbent core is made from viscose acetate, polyester and/or polypropylene.

The fibrous material of the fluid-absorbent core may be uniformly mixed to generate a homogenous or inhomogenous fluid-absorbent core. Alternatively the fibrous material may be concentrated or laid in separate layers optionally comprising fluid-absorbent polymer material. Suitable storage layers of the fluid-absorbent core comprising homogenous mixtures of fibrous materials comprising fluid-absorbent polymer material. Suitable storage layers of the fluid-absorbent core including a layered core-system comprise homogenous mixtures of fibrous materials and comprise fluid-absorbent polymer material, whereby each of the layers may be built from any fibrous material by means known in the art. The sequence of the layers may be directed such that a desired fluid acquisition, distribution and transfer results, depending on the amount and distribution of the inserted fluid-absorbent material, e.g. fluid-absorbent polymer particles. Preferably there are discrete zones of highest absorption rate or retention within the storage layer of the fluid-absorbent core, formed of layers or inhomogenous mixtures of the fibrous material, acting as a matrix for the incorporation of fluid-absorbent polymer particles. The zones may extend over the full area or may form only parts of the fluid-absorbent core.

Suitable fluid-absorbent cores comprise fibrous material and fluid-absorbent material. Suitable is any fluid-absorbent material that is capable of absorbing and retaining body fluids or body exudates such as cellulose wadding, modified and unmodified cellulose, crosslinked cellulose, laminates, composites, fluid-absorbent foams, materials described as in the chapter “Liquid-pervious Layer (A)” above, fluid-absorbent polymer particles and combinations thereof.

Typically the fluid-absorbent cores may contain a single type of fluid-absorbent polymer particles or may contain fluid-absorbent polymer particles derived from different kinds of fluid-absorbent polymer material. Thus, it is possible to add fluid-absorbent polymer particles from a single kind of polymer material or a mixture of fluid-absorbent polymer particles from different kinds of polymer materials, e.g. a mixture of regular fluid-absorbent polymer particles, derived from gel polymerization with fluid-absorbent polymer particles, derived from droplet polymerization. Alternatively it is possible to add fluid-absorbent polymer particles derived from inverse suspension polymerization.

Alternatively it is possible to mix fluid-absorbent polymer particles showing different feature profiles. Thus, the fluid-absorbent core may contain fluid-absorbent polymer particles with uniform pH value, or it may contain fluid-absorbent polymer particles with different pH values, e.g. two- or more component mixtures from fluid-absorbent polymer particles with a pH in the range from about 4.0 to about 7.0. Preferably, applied mixtures deriving from mixtures of fluid-absorbent polymer particles got from gel polymerization or inverse suspension polymerization with a pH in the range from about 4.0 to about 7.0 and fluid-absorbent polymer particles got from droplet polymerization.

Suitable fluid-absorbent cores are also manufactured from loose fibrous materials by adding fluid-absorbent particles and/or fluid-absorbent polymer fibers or mixtures thereof. The fluid-absorbent polymer fibers may be formed from a single type of fluid-absorbent polymer fiber or may contain fluid-absorbent polymer fibers from different polymeric materials. The addition of fluid-absorbent polymer fibers may be preferred for being distributed and incorporated easily into the fibrous structure and remaining better in place than fluid-absorbent polymer particles. Thus, the tendency of gel blocking caused by contacting each other is reduced. Further, fluid-absorbent polymer fibers are softer and more flexible.

In the process of manufacturing the fluid-absorbent core, fluid-absorbent polymer particles and/or fluid-absorbent fibers are brought together with structure forming compounds such as fibrous matrices. Thus, the fluid-absorbent polymer particles and/or fluid-absorbent fibers may be added during the process of forming the fluid-absorbent core from loose fibers. The fluid-absorbent core may be formed by mixing fluid-absorbent polymer particles and/or fluid-absorbent fibers with fibrous materials of the matrix at the same time or adding one component to the mixture of two or more other components either at the same time or by continuously adding.

Suitable fluid-absorbent cores including mixtures of fluid-absorbent polymer particles and/or fluid-absorbent fibers and fibrous material building matrices for the incorporation of the fluid-absorbent material. Such mixtures can be formed homogenously, that is all components are mixed together to get a homogenous structure. The amount of the fluid-absorbent materials may be uniform throughout the fluid-absorbent core, or may vary, e.g. between the central region and the distal region to give a profiled core concerning the concentration of fluid-absorbent material. Suitable fluid absorbent cores are described e.g. in WO 2010002828 A1, WO 2004 073571 and WO 2010 133529.

Techniques of application of the fluid-absorbent polymer materials into the absorbent core are known to persons skilled in the art and may be volumetric, loss-in-weight or gravimetric. Known techniques include the application by vibrating systems, single and multiple auger systems, dosing roll, weigh belt, fluid bed volumetric systems and gravitational sprinkle and/or spray systems. Further techniques of insertion are falling dosage systems consensus and contradictory pneumatic application or vacuum printing method of applying the fluid absorbent polymer materials.

Suitable fluid-absorbent cores may also include layers, which are formed by the process of manufacturing the fluid-absorbent article. The layered structure may be formed by subsequently generating the different layers in height (z-direction).

Alternatively a core-structure can be formed from two or more preformed layers to get a layered fluid-absorbent core. The layers may have different concentrations of fluid-absorbent polymer material showing concentrations in the range from about 20 to 95%. These uniform or different layers can be fixed to each other at their adjacent plane surfaces. Alternatively, the layers may be combined in a way that a plurality of chambers are formed, in which separately fluid-absorbent polymer material is incorporated.

Suitable preformed layers are processed as e.g. air-laid, wet-laid, laminate or composite structure.

Alternatively layers of other materials can be added, e.g. layers of opened or closed celled foams or perforated films. Included are also laminates of at least two layers comprising said fluid-absorbent polymer material.

Further a composite structure can be formed from a carrier layer (e.g. a polymer film), onto which the fluid-absorbent polymer material is affixed. The fixation can be done at one side or at both sides. The carrier layer may be pervious or impervious for body-fluids.

Alternatively, it is possible to add monomer solution after the formation of a layer or onto a carrier layer and polymerize the coating solution by means of UV-induced polymerization technologies. Thus, “in situ”-polymerization is a further method for the application of fluid-absorbent polymers.

Thus, suitable fluid-absorbent cores comprising at least 60% by weight fluid-absorbent polymer particles and not more than 40% % by weight of cellulose based fibers, preferably at least 70% by weight fluid-absorbent polymer particles and not more than 30% by weight of cellulose based fibers, more preferably 80% by weight fluid-absorbent polymer particles and not more than 20% by weight of cellulose based fibers, most preferably at least 90% by weight fluid-absorbent polymer particles and not more than 10% by weight of cellulose based fibers, based on the fluid-absorbent core.

The quantity of fluid-absorbent polymer particles and/or fluid-absorbent fibers within the fluid-absorbent core is from 3 to 20 g, preferably from 6 to 14 g, and from 8 to 12 g in the case of maxi-diapers, and in the case of incontinence products up to about 50 g.

Typically fluid-absorbent articles comprising at least an upper liquid-pervious layer (A), at least a lower liquid-impervious layer (B) and at least one fluid-absorbent core between the layer (A) and the layer (B) besides other optional layers. In order to increase the control of body fluid absorption and/or to increase the flexibility in the ratio weight percentages of fluid-absorbent polymer particles to fibrous matrix it may be advantageous to add one or more further fluid-absorbent cores. The addition of a second fluid-absorbent core to the first fluid-absorbent core offers more possibilities in body fluid transfer and distribution. Moreover higher quantities of discharged body fluids can be retained. Having the opportunity of combining several layers showing different fluid-absorbent polymer concentration and content, it is possible to reduce the thickness of the fluid-absorbent article to a minimum even if there are several fluid-absorbent cores included.

Suitable fluid-absorbent cores may be formed from any material known in the art which is designed to acquire, transfer, and retain discharged body fluids. The technology of manufacturing may also be anyone known in the art. Preferred technologies include the application of monomer-solution to a transported fibrous matrix and thereby polymerizing, known as in-situ technology, or the manufacturing of air-laid composites.

Suitable fluid-absorbent articles are including single or multi-core systems in any combination with other layers which are typically found in fluid-absorbent articles. Preferred fluid-absorbent articles include single- or double-core systems; most preferably fluid-absorbent articles include a single fluid-absorbent core.

The fluid-absorbent core typically has a uniform size or profile. Suitable fluid-absorbent cores can also have profiled structures, concerning the shape of the core and/or the content of fluid-absorbent polymer particles and/or the distribution of the fluid-absorbent polymer particles and/or the dimensions of the different layers if a layered fluid-absorbent core is present.

It is known that absorbent cores providing a good wet immobilization by combining several layers, e.g. a substrate layer, layers of fluid-absorbent polymer and layers of thermoplastic material. Suitable absorbent cores may also comprise tissue or tissue laminates. Known in the art are single or double layer tissue laminates formed by folding the tissue or the tissue laminate onto itself.

These layers or foldings are preferably joined to each e.g. by addition of adhesives or by mechanical, thermal or ultrasonic bonding or combinations thereof. Fluid-absorbent polymer particles may be comprised within or between the individual layers, e.g. by forming separate fluid-absorbent polymer layers.

Thus, according to the number of layers or the height of a voluminous core, the resulting thickness of the fluid-absorbent core will be determined. Thus, fluid-absorbent cores may be flat as one layer (plateau) or have three-dimensional profile.

Generally the upper liquid-pervious layer (A) and the lower liquid-impervious layer (B) may be shaped and sized according to the requirements of the various types of fluid-absorbent articles and to accommodate various user/wearer's size. Thus, the combination of the upper liquid-pervious layer and the lower liquid-impervious layer may have all dimensions or shapes known in the art. Suitable combinations have an hourglass shape, rectangular shape, trapezoidal shape, t- or double t-shape or showing anatomical dimensions.

The fluid-absorbent core may comprise additional additives typically present in fluid-absorbent articles known in the art. Exemplary additives are fibers for reinforcing and stabilizing the fluid-absorbent core. Preferably polyethylene is used for reinforcing the fluid-absorbent core.

Further suitable stabilizers for reinforcing the fluid-absorbent core are materials acting as binder.

In varying the kind of binder material or the amount of binder used in different regions of the fluid-absorbent core it is possible to get a profiled stabilization. For example, different binder materials exhibiting different melting temperatures may be used in regions of the fluid-absorbent core, e.g. the lower melting one in the central region of the core, and the higher melting in the distal regions. Suitable binder materials may be adhesive or non-adhesive fibers, continuously or discontinuously extruded fibers, bi-component staple fibers, non-elastomeric fibers and sprayed liquid binder or any combination of these binder materials.

Further, thermoplastic compositions usually are added to increase the integrity of the core layer. Thermoplastic compositions may comprise a single type of thermoplastic polymers or a blend of thermoplastic polymers. Alternatively, the thermoplastic composition may comprise hot melt adhesives comprising at least one thermoplastic polymer together with thermoplastic diluents such as tackifiers, plasticizers or other additives, e.g. antioxidants. The thermoplastic composition may further comprise pressure sensitive hot melt adhesives comprising e.g. crystalline polypropylene and an amorphous polyalphaolefin or styrene block copolymer and mixture of waxes.

Suitable thermoplastic polymers are styrenic block copolymers including A-B-A triblock segments, A-B diblock segments and (A-B)ₙ radial block copolymer segments. The letter A designs non-elastomeric polymer segments, e.g. polystyrene, and B stands for unsaturated conjugated diene or their (partly) hydrogenated form. Preferably B comprises isoprene, butadiene, ethylene/butylene (hydrogenated butadiene), ethylene/propylene (hydrogenated isoprene) and mixtures thereof.

Other suitable thermoplastic polymers are amorphous polyolefins, amorphous polyalphaolefins and metallocene polyolefins.

Concerning odor control, perfumes and/or odor control additives are optionally added. Suitable odor control additives are all substances of reducing odor developed in carrying fluid-absorbent articles over time known in the art. Thus, suitable odor control additives are inorganic materials, such as zeolites, activated carbon, bentonite, silica, aerosile, kieselguhr, clay; chelants such as ethylenediamine tetraacetic acid (EDTA), cyclodextrins, aminopolycarbonic acids, ethylenediamine tetramethylene phosphonic acid, aminophosphate, polyfunctional aromates, N,N-disuccinic acid.

Suitable odor control additives are further antimicrobial agents such as quaternary ammonium, phenolic, amide and nitro compounds and mixtures thereof; bactericides such as silver salts, zinc salts, cetylpyridinium chloride and/or triclosan as well as surfactants having an HLB value of less than 12.

Suitable odor control additives are further compounds with acid groups such as ascorbic, benzoic, citric, salicylic or sorbic acid and fluid-soluble polymers of monomers with acid groups, homo- or co-polymers of C₃-C₅ mono-unsaturated carboxylic acids.

Suitable odor control additives are further perfumes such as allyl caproate, allyl cyclo-hexaneacetate, allyl cyclohexanepropionate, allyl heptanoate, amyl acetate, amyl propionate, anethol, anixic aldehyde, anisole, benzaldehyde, benzyl acetete, benzyl acetone, benzyl alcohole, benzyl butyrate, benzyl formate, camphene, camphor gum, laevo-carveol, cinnamyl formate, cis-jasmone, citral, citronellol and its derivatives, cuminic alcohol and its derivatives, cyclal C, dimethyl benzyl carbinol and its derivatives, dimethyl octanol and its derivatives, eucalyptol, geranyl derivatives, lavandulyl acetete, ligustral, d-limonene, linalool, linalyl derivatives, menthone and its derivatives, myrcene and its derivatives, neral, nerol, p-cresol, p-cymene, orange terpenes, alpha-ponene, 4-terpineol, thymol etc.

Masking agents are also used as odor control additives. Masking agents are in solid wall material encapsulated perfumes. Preferably, the wall material comprises a fluid-soluble cellular matrix which is used for time-delay release of the perfume ingredient.

Further suitable odor control additives are transition metals such as Cu, Ag, Zn; enzymes such as urease-inhibitors, starch, pH buffering material, chitin, green tea plant extracts, ion exchange resin, carbonate, bicarbonate, phosphate, sulfate or mixtures thereof.

Preferred odor control additives are green tea plant extracts, silica, zeolite, carbon, starch, chelating agent, pH buffering material, chitin, kieselguhr, clay, ion exchange resin, carbonate, bicarbonate, phosphate, sulfate, masking agent or mixtures thereof. Suitable concentrations of odor control additives are from about 0.5 to about 300 gsm.

The bulk density of the fluid-absorbent core is in the range of 0.12 to 0.35 g/cm³. The thickness (z-dimension) of the fluid-absorbent core is in the case of diapers in the range of 1 to 6 mm, preferably 1.5 to 3 mm, in the case of incontinence products in the range of 3 to 15 mm.

### 3. Optional Dusting Layer

An optional component for inclusion into the absorbent core is a dusting layer adjacent to. The dusting layer is a fibrous layer and may be placed on the top and/or the bottom of the absorbent core. Typically, the dusting layer is underlying the storage layer. This underlying layer is referred to as a dusting layer, since it serves as carrier for deposited fluid-absorbent polymer particles during the manufacturing process of the fluid-absorbent core. If the fluid-absorbent polymer material is in the form of macrostructures, films or flakes, the insertion of a dusting layer is not necessary. In the case of fluid-absorbent polymer particles derived from droplet polymerization, the particles have a smooth surface with no edges. Also in this case, the addition of a dusting layer to the fluid-absorbent core is not necessary. On the other side, as a great advantage the dusting layer provides some additional fluid-handling properties such as wicking performance and may offer reduced incidence of pin-holing and or pock marking of the liquid-impervious layer (B).

Preferably, the dusting layer is a fibrous layer comprising fluff (cellulose based fibers), most preferably the dusting layer is non-cellulose based material such as spun-melt-spun (SMS), spun-bond, SMMS combinations and thermalbond polypropylene contacing the formation area and/or marrying the fluid absorbent immediately upon exit from the forming chamber before compression. Hot-melt adhesive is also employed to bond the non-cellulose fibre dusting layer to the core and/or bonding between the non-cellulose fibre based dusting following lamination or wrapping techniques know to people skilled in the art.

IV. Acquisition-Distribution Layer (D)

The acquisition-distribution layer (D) is located between the upper layer (A) and the fluid-absorbent core (C) and is preferably constructed to efficiently acquire discharged body fluids and to transfer and distribute them to other regions of the fluid-absorbent core, where the body fluids are immobilized and stored. Thus, the upper layer transfers the discharged liquid to the acquisition-distribution layer (D) for distributing it to the fluid-absorbent core.

In case of diapers the length of the acquisition-distribution layer is in its longitudinal direction shorter than the fluid-absorbent core. The length of the acquisition-distribution layer is in its longitudinal direction typically at least 50%, preferred at least 60%, more preferred at least 62.5% of the length of the fluid-absorbent core.

Typically the acquisition-distribution layer is not centered on the fluid-absorbent core. The distance between the centers of the fluid-absorbent core and the acquisition-distribution layer is typically from 5 to 20%, preferably from 8 to 18%, more preferably from 9 to 17% most preferred 10 to 16% of the total length of the fluid-absorbent core.

A typical acquisition-distribution layer may comprise a high loft synthetic fiber carded web which may be further bonded by air, calendaring and/or other modifications e.g. resin additive.

The acquisition-distribution layer comprises fibrous material and optionally fluid-absorbent polymer particles.

The fibrous material may be hydrophilic, hydrophobic or can be a combination of both hydrophilic and hydrophobic fibers. It may be derived from synthetic non-cellulose based fibers alone or in combination with not more than 10% by weight of natural cellulose based fibers, based on the sum of synthetic non-cellulose based fibers and cellulose based fibers.

Suitable acquisition-distribution layers are formed from synthetics alone, or synthetics in combination with not more than 10% by weight of cellulose based fibers and/or modified cellulose based fibers, based on the sum of synthetic non-cellulose based fibers and cellulose based fibers.

Multiple fiber types and combinations thereof can be employed, for example polyester, co-polyester, polypropylene along with optimization of the fiber dtex, preferred are e.g. 6-7 dtex for polyester and for copolyester with basis weight of 40-60 gsm, or for light incontinence acquisition-distribution layer e.g. a bi-component fibrous web of polypropylene and polyethylene with 3.3 dtex and 3.2 dtex respectively.

Examples of further suitable hydrophilic, hydrophobic fibers especially synthetic non-cellulose based fibers, as well as modified or unmodified natural cellulose based fibers are given in the chapter “Liquid-pervious Layer (A)” above.

For providing improved fluid acquisition and distribution properties suitable acquisition-distribution layers according to the invention comprise synthetic non-cellulose based fibers and optionally cellulose based fibers, whereas modified cellulose based fibers are preferred.

Examples for modified cellulose based fibers are chemically treated cellulose based fibers, especially chemically stiffened cellulose based fibers. The term “chemically stiffened cellulose based fibers” means cellulose based fibers that have been stiffened by chemical means to increase the stiffness of the fibers. Such means include the addition of chemical stiffening agent in the form of coatings and impregnates. Suitable polymeric stiffening agents can include: cationic modified starches having nitrogen containing groups, latexes, wet strength resins such as polyamide-epichlorohydrin resin, polyacrylamide, urea formaldehyde and melamine formaldehyde resins and polyethylenimine resins.

Stiffening may also include altering the chemical structure, e.g. by crosslinking polymer chains. Thus crosslinking agents can be applied to the fibers that are caused to chemically form intrafiber crosslink bonds. Further cellulose based fibers may be stiffened by crosslink bonds in individualized form. Suitable chemical stiffening agents are typically monomeric crosslinking agents including C₂-C₈ dialdehyde, C₂-C₈ monoaldehyde having an acid functionality, and especially C₂-C₉ polycarboxylic acids.

Preferably the modified cellulose based fibers are chemically treated cellulose based fibers.

Examples of synthetic non-cellulose based fibers are found in the Chapter “Liquid-pervious Layer (A)” above.

Hydrophilic synthetic non-cellulose based fibers are preferred.

Especially preferred are polyester, polyethylene, polypropylene, polylactic acid, polyamides and/or blends thereof.

Hydrophilic synthetic non-cellulose based fibers may be obtained by chemical modification of hydrophobic fibers. Preferably, hydrophilization is carried out by surfactant treatment of hydrophobic fibers. Thus the surface of the hydrophobic fiber can be rendered hydrophilic by treatment with a nonionic or ionic surfactant, e.g., by spraying the fiber with a surfactant or by dipping the fiber into a surfactant. Further preferred are permanent hydrophilic synthetic fibers.

The fibrous material of the acquisition-distribution layer may be fixed to increase the strength and the integrity of the layer. Technologies for consolidating fibers in a web are mechanical bonding, thermal bonding and chemical bonding. Detailed description of the different methods of increasing the integrity of the web is given in the Chapter “Liquid-pervious Layer (A)” above.

Suitable acquisition-distribution layers may comprise fibrous material and fluid-absorbent polymer particles distributed within. The fluid-absorbent polymer particles may be added during the process of forming the layer from loose fibers, or, alternatively, it is possible to add monomer solution after the formation of the layer and polymerize the coating solution by means of UV-induced polymerisation technologies. Thus, “in situ”-polymerisation is a further method for the application of fluid-absorbent polymers.

V. Optional Tissue Layer (E)

An optional tissue layer is disposed immediately above and/or below (C).

The material of the tissue layer may comprise any known type of substrate, including webs, garments, textiles and films. The tissue layer may comprise natural cellulose based fibers, such as cotton, flax, linen, hemp, wool, silk, fur, hair and naturally occurring mineral fibers. The tissue layer may also comprise synthetic non-cellulose based fibers such as rayon and lyocell (derived from cellulose), polysaccharides (starch), polyolefin fibers (polypropylene, polyethylene), polyamides, polyester, butadiene-styrene block copolymers, polyurethane and combinations thereof. Preferably, the tissue layer comprises cellulose based fibers. The optional tissue layer may be ‘open’ allowing passage of air through the substrate or ‘closed’ not allowing air passage through the substrate material.

VI. Other Optional Components (F)

### 1. Leg Cuff

Typical leg cuffs comprising nonwoven materials which can be formed by direct extrusion processes during which the fibers and the nonwoven materials are formed at the same time, or by laying processes of preformed fibers which can be laid into nonwoven materials at a later point of time. Examples for direct extrusion processes include spunbonding, meltblowing, solvent spinning, electrospinning and combinations thereof. Examples of laying processes include wet-laying and dry-laying (e.g. air-laying, carding) methods. Combinations of the processes above include spunbond-meltblown-spunbond (sms), spunbond-meltblow-meltblown-spunbond (smms), spunbond-carded (sc), spunbond-airlaid (sa), meltblown-airlaid (ma) and combinations thereof. The combinations including direct extrusion can be combined at the same point in time or at a subsequent point in time. In the examples above, one or more individual layers can be produced by each process. Thus, “sms” means a three layer nonwoven material, “smsms” or “ssmms” means a five layer nonwoven material. Usually, small type letters (sms) designate individual layers, whereas capital letters (SMS) designate the compilation of similar adjacent layers.

Further, suitable leg cuffs are provided with elastic strands.

Preferred are leg cuffs from synthetic non-cellulose based fibers showing the layer combinations sms, smms or smsms. Preferred are nonwovens with the density of 7 to 17 gsm. Preferably leg cuffs are provided with two elastic strands.

### 2. Elastics

The elastics are used for securely holding and flexibly closing the fluid-absorbent article around the wearer's body, e.g. the waist and the legs to improve containment and fit. Leg elastics are placed between the outer and inner layers or the fluid-absorbent article, or between the outer cover and the bodyside liner. Suitable elastics comprising sheets, ribbons or strands of thermoplastic polyurethane, elastomeric materials, poly(ether-amide) block copolymers, thermoplastic rubbers, styrene-butadiene copolymers, silicon rubbers, natural rubbers, synthetic rubbers, styrene isoprene copolymers, styrene ethylene butylene copolymers, nylon copolymers, spandex fibers comprising segmented polyurethane and/or ethylene-vinyl acetate copolymer. The elastics may be secured to a substrate after being stretched, or secured to a stretched substrate. Otherwise, the elastics may be secured to a substrate and then elasticized or shrunk, e.g. by the application of heat.

### 3. Closure System

The closure system include tape tabs, landing zone, elastomerics, pull ups with refastenable side sections and the belt system.

At least a part of the first waist region is attached to a part of the second waist region by the closing system to hold the fluid-absorbent article in place and to form leg openings and the waist of the fluid-absorbent article. Preferably the fluid-absorbent article is provided with a re-closable closing system.

The closing system is either re-sealable or permanent, including any material suitable for such a use, e.g. plastics, elastics, films, foams, nonwoven substrates, woven substrates, paper, tissue, laminates, fiber reinforced plastics and the like, or combinations thereof. Preferably the closing system includes flexible materials and works smooth and softly without irritating the wearer's skin.

One part of the closing elements is an adhesive tape, or comprises a pair of laterally extending tabs disposed on the lateral edges of the first waist region. Tape tabs are typically attached to the front body panel and extend laterally from each corner of the first waistband. These tape tabs include an adhesive inwardly facing surface which is typically protected prior to use by a thin, removable cover sheet.

Suitable tape tabs may be formed of thermoplastic polymers such as polyethylene, polyurethane, polystyrene, polycarbonate, polyester, ethylene vinyl acetate, ethylene vinyl alcohol, ethylene vinyl acetate, acrylate or ethylene acrylic acid copolymers.

Suitable closing systems comprise further a hook portion of a hook and loop fastener and the target devices comprise the loop portion of a hook and loop fastener.

Suitable mechanical closing systems include a landing zone. Mechanical closing sys-terns may fasten directly into the outer cover. The landing zone acts as an area of the fluid-absorbent article into which it is desirable to engage the tape tabs. The base material may include a loop material. The loop material may include a backing material and a layer of a non-woven spunbond web attacked to the backing material.

Thus suitable landing zones can be made by spunbonding, spunbonded nonwovens are made from melt-spun fibers formed by extruding molten thermoplastic material. An example is bi-oriented polypropylene (BOPP), most preferred are brushed/closed loop in the case of prevalent mechanical closure systems.

Further, suitable mechanical closing systems including elastic units serving as a flexible waist band or side panels for fluid-absorbents articles, such as pants or pull-ups. The elastic units enable the wearer to pull down the fluid-absorbent article as e.g. a training pant or mobile user adult incontinence fluid absorbent article.

Suitable pants-shaped fluid-absorbent article has front section, rear section, crotch section, side sections for connecting the front and rear sections in lateral direction, hip section, elastic waist region and liquid-tight outer layer. The hip section is arranged around the waist of the user. The disposable pants-shaped fluid-absorbent article (pull-up) has favorable flexibility, stretchability, leak-proof property and fit property, hence imparts excellent comfort to the wearer.

Suitable pull-ups comprising thermoplastic films, sheets and laminates have a low modulus, good tear strength and high elastic recovery.

Suitable closing systems may further comprise elastomerics for the production of elastic areas within the fastening devices of the fluid-absorbent article. Elastomerics provide a conformable fit of the fluid-absorbent article to the wearer at the waist and leg openings, while maintaining adequate performance against leakage.

Suitable elastomerics are elastomeric polymers or elastic adhesive materials showing vapor permeability and liquid barrier properties. Preferred elastomerics are retractable after elongation to a length equivalent to its original length.

Suitable closing systems further comprise a belt system, comprising waist-belt and leg-belts for flexibly securing the fluid-absorbent article on the body of the wearer and to provide an improved fit on the wearer. Suitable waist-belts comprise two elastic belts, a left elastic belt, and a right elastic belt. The left elastic belt is associated with each of the left angular edges. The right elastic belt associated with each of the right angular edges. The left and right side belts are elastically extended when the absorbent garment is laid flat. Each belt is connected to and extends between the front and rear of the fluid-absorbent article to form a waist hole and leg holes.

Preferably the belt system is made of elastomeric material, thus providing a conformable fit of the fluid-absorbent article and maintaining adequate performance against leakage.

### D. Fluid-Absorbent Article Construction

The present invention further relates to the joining of the components and layers, films, sheets, tissues or substrates mentioned above to provide the fluid-absorbent article. At least two, preferably all layers, films, sheets, tissues or substrates are joined.

Suitable fluid-absorbent articles include a single- or multiple fluid-absorbent core-system. Preferably fluid-absorbent articles include a single fluid-absorbent core-system.

Suitable fluid-storage layers of the fluid-absorbent core comprising homogenous or inhomogenous mixtures of fibrous materials comprising fluid-absorbent polymer particles homogenously or inhomogenously dispersed in it.

Suitable fluid-storage layers of the fluid-absorbent core including a layered fluid-absorbent core-system comprising homogenous mixtures of fibrous materials and optionally comprising fluid-absorbent polymer particles, whereby each of the layers may be prepared from any fibrous material by means known in the art.

Preferably the fluid-absorbent core comprises at least 60% by weight of fluid-absorbent polymer particles and not more than 40% by weight of cellulose based fibers, based on the sum of fluid-absorbent polymer particles and cellulose based fibers.

According to the invention it is preferred, that the fluid-absorbent core is covered by an acquisition-distribution layer comprising at least 90% by weight of synthetic non-cellulose based fibers and not more than 10% by weight of cellulose based fibers, based on the sum of synthetic non-cellulose based fibers and cellulose based fibers.

Preferably the acquisition-distribution layer is in longitudinal direction asymmetric positioned on the fluid-absorbent core.

Especially preferred are fluid-absorbent articles having a diaper construction as explained above, wherein the acquisition-distribution layer is essentially free of cellulose based fibers.

It is preferred, that the thickness (z-dimension) of the acquisition-distribution layer is not more than 60% of the thickness of the fluid-absorbent core and the thickness deviation of the bi-folded fluid-absorbent article in longitudinal direction is less than 10%.

Preferable the thickness of the unfolded fluid-absorbent article is less than 3 mm.

The distance between the centers of the fluid-absorbent core and the acquisition-distribution layer is from 5 to 20%, preferably from 10 to 16% of the total length of the fluid-absorbent core.

It is preferred that the synthetic non-cellulose based fibers are based on polyester, polyethylene, polypropylene, polylactic acid, polyamide and/or blends thereof.

Furthermore the fluid-absorbent core may be encapsulated by wrapping with a nonwoven material or a tissue paper.

The fluid-absorbent core comprises at least 80% by weight of water-absorbent polymer particles and less than 10% by weight of cellulose based fibers.

It is preferred, e.g. for a homogeneous distribution of the water-absorbent polymer particles to place them in discrete regions of the fluid-absorbent core.

The amount of water-absorbent polymer particles included in the absorbent core is at least 8 g. The particles preferably have a bulk density of at least 0.55 g/cm³ and a centrifuge retention capacity of at least 24 g/g, an absorbency under high load of at least 18 g/g and a saline flow conductivity of at least 20×10⁻⁷ cm³s/g.

In order to immobilize the fluid-absorbent polymer particles, the adjacent layers are fixed by the means of thermoplastic materials, thereby building connections throughout the whole surface or alternatively in discrete areas of junction.

For the latter case, cavities or pockets are built carrying the fluid-absorbent particles. The areas of junction may have a regular or irregular pattern, e.g. aligned with the longitudinal axis of the fluid-absorbent core or in a pattern of polygons, e.g. pentagons or hexagons. The areas of junction itself may be of rectangular, circular or squared shape with diameters between about 0.5 mm and 2 mm. Fluid-absorbent articles comprising areas of junction show a better wet strength.

The construction of the products fluid-absorbent core and the components contained therein is made and controlled by the discrete application of hotmelt adhesives as known to people skilled in the art. Examples would be e.g. Dispomelt 505B, Dispomelt Cool 1 101, as well as other specific function adhesives manufactured by for example Henkel, Fuller, Colchimica or Bostik.

Fluid-absorbent articles according to the invention comprise an indicator substance which changes color as a result of contact with proteins. The indicator substance is applied to a carrier material such as e.g. the inside, the side adjacent to the absorbent core, of the liquid impervious layer or any one of the upper liquid-pervious layer, the lower liquid-impervious layer, the fluid-absorbent core and the additional layer or the fluid-absorbent particles or in another embodiment a layer, a membrane or a strip fastened preferably on the inside of the liquid impervious layer.

According to the invention the carrier material is preferably treated with a ninhydrin solution e.g. a ethanolic ninhydrin solution to apply the ninhydrin on the respective carrier material.

According to the invention the indicator can be applied in distinct zones on the carrier material.

In an embodiment according to the invention the indicator-substance is applied directly on the carrier material. It is further preferred to apply the indicator-substance indirectly by e.g. mixing with adhesives, varnishes, coatings, printing inks.

The indicator may be applied on the carrier material in patterns such as dots, circles, squares, triangles, lines, pictogrammes or a combination thereof.

Furthermore the carrier material may be water-absorbent polymer particles wherein the indicator may be dispersed within the water-absorbent polymer particles such that the particles entrap the indicator.

A carrier material according to the invention is a supporting structure for carrying the indicator. Useful carrier materials are disclosed in WO2009/005884, such as fluff, flexible ceramic sheets, films, woven or knitted materials, nonwovens, paper, tissue, foams, sponges or membranes or a variety of combinations thereof. Preferred carrier material is white or transparent and the indicator has a good adhesion to the surface of the material. In a preferred embodiment the material is porous.

The carrier material may be made from natural material or a polymer based material, or a combination of natural or polymer based materials.

One suitable material is a nonwoven web of fibers, wherein the fibers can be synthetic polymers, natural or a combination of synthetic or natural. Synthetic polymers include e.g. polyesters, polyamides, polyimides, nylon, polyolefines or combinations thereof.

Furthermore the material may be a hydrogel such as agarose gel, polyacrylamides, polysiloxanes, polyacrylates preferably in bead or fiber form.

According to the present invention membranes are preferred, especially those for water filtration. Such membranes are e.g. polyethersulfone membranes with surfaces of different porosity, e.g. with a dense and an open surface.

According to the invention membranes with a molecular weight cutoff (MWCO) of 10.000.000 Da are suitable, preferably of 1.000.000 Da, more preferably of 100.000 Da most preferably with a molecular weight cutoff of 100.000 Da.

Useful membranes are disclosed in the monograph “Membranen: Grundlagen, Verfahren and industrielle Anwendungen” Klaus Ohlrogge, Katrin Ebert, Wiley-VCH, April 2006.

Membranes according to the invention are preferably hydrophilic. The liquid content of the feces and/or the urine is very quickly absorbed by a hydrophilic membrane.

Furthermore the membranes preferably consisting of polymers having a molecular weight of 60.000 to 80.000 Da.

According to the invention the color change is visible also on the side of the carrier material, e.g. of the membrane not in direct contact with the feces and/or urine.

Wherein it is preferred that the indicator substance is applied in such a way, that in case of the presence of feces especially of newborn and breast-feeded babies and/or substances present in urine indicating kidney or vascular diseases the color-change of the indicator substance should be visible through the material of the liquid impervious layer of the fluid absorbent article.

According to the invention it is preferred that at least one part of the liquid-impervious layer having a color or transparency different from the rest of the layer.

Therefore it is preferred that the indicator is applied on or adjacent to the at least one part of the liquid impervious layer having a color or transparency allowing the color change of the indicator-substance to be visible through the impervious layer material.

### Methods

The measurements should unless stated otherwise, be carried out at an ambient temperature of 23±2° C. and an atmospheric humidity of 50±10%. The fluid absorbent polymers are mixed thoroughly before the measurement.

Density of the fluid-absorbent polymer particles

The apparent density, also known as bulk density, of the absorbent polymer material, typically in particle form, can be measured according to the standard INDA-EDANA test method WSP 260.2 (05), wherein the test conditions, referred to under Section 6.2 of the standard test method, are to be set as 23±2° C. and a humidity of 50±5%.

### Saline Flow Conductivity (SFC)

The saline flow conductivity is, as described in EP 0 640 330 A1, determined as the gel layer permeability of a swollen gel layer of fluid-absorbent polymer particles, although the apparatus described on page 19 and in FIG. 8 in the aforementioned patent application was modified to the effect that the glass frit (40) is no longer used, the plunger (39) consists of the same polymer material as the cylinder (37) and now comprises 21 bores having a diameter of 9.65 mm each distributed uniformly over the entire contact surface. The procedure and the evaluation of the measurement remains unchanged from EP 0 640 330 A1. The flow rate is recorded automatically.

The saline flow conductivity (SFC) is calculated as follows:

SFC[cm³s/g]=(Fg(t=0)×L0)/(d×A×WP),

where Fg(t=0) is the flow rate of NaCl solution in g/s, which is obtained by means of a linear regression analysis of the Fg(t) data of the flow determinations by extrapolation to t=0, LO is the thickness of the gel layer in cm, d is the density of the NaCl solution in g/cm³, A is the surface area of the gel layer in cm² and WP is the hydrostatic pressure over the gel layer in dyn/cm².

### Centrifuge Retention Capacity (CRC)

The centrifuge retention capacity of the fluid-absorbent polymer particles is determined by the EDANA recommended test method No. WSP 241.3-10 “Centrifuge Retention Capacity”, wherein for higher values of the centrifuge retention capacity larger tea bags have to be used due to bursting of the tea-bag upon hydration.

### Free Swell Gel Bed Permeability (GBP)

The method for determination of the Free Swell Gel Bed Permeability (Free Swell GBP) is described in US patent application no. US 2005/0256757 A1, paragraphs [0061] to

### Absorbency Under High Load (AUHL)

The absorbency under high load of the fluid-absorbent polymer particles is determined analogously to the EDANA recommended test method No. WSP 242.3-10 “Absorption Under Pressure”, except using a weight of 49.2 g/cm² instead of a weight of 21.0 g/cm².

### Moisture Content

The moisture content of the fluid-absorbent polymer particles is determined by the EDANA recommended test method No. WSP 230.3-10 “Moisture Content”.

### Residual Monomers

The level of residual monomers in the fluid-absorbent polymer particles is determined by the EDANA recommended test method No. WSP 210.3-10 “Residual Monomers”.

### Particle Size Distribution

The particle size distribution of the fluid-absorbent polymer particles is determined with EDANA recommended test method No. 220.3.10 “Particle size distribution by sieve fractionation”

### Extractables

The level of extractable constituents in the fluid-absorbent polymer particles is determined by the EDANA recommended test method No. WSP 270.2-05 “Extractables”.

The EDANA test methods are obtainable, for example, from the EDANA, Avenue Eugene Plasky 157, B-1030 Brussels, Belgium.

### EXAMPLES

### General Procedure for Preparation of PES Flat Sheet Membranes

Into a three neck flask equipped with a magnetic stirrer there is added 80 ml of N-methylpyrrolidone (NMP), 5 g of polyvinylpyrrolidone (PVP, Luvitec® K90, BASF SE) and 15 g of polyethersulfone (PES, Ultrason® E 3010P, BASF SE). The mixture is heated under gentle stirring at 60° C. until a homogeneous clear viscous solution is obtained.

The solution is degassed overnight at room temperature. After that the membrane solution is reheated at 60° C. for 2 hours and casted onto a glass plate with a casting machine (Erichsen cast master 510) at 200 microns wet thickness and 40° C., casting speed: 5 mm/sec.

The membrane film is allowed to rest for 30 seconds before immersion in a water bath at 25° C. for 10 minutes.

After rinsing and removal of excess PVP, a flat sheet continuous film with micro structural characteristics of UF membranes having dimension of at least 20×30 cm size is obtained. The membrane presents a top thin skin layer (1-3 microns) and a porous layer underneath (thickness: 100-150 microns).

### Example 1

### Membrane Treatment

1.78 g Ninhydrin (Ninhydrin; Fluka; Art.Nr. 72490-25G; ACS reagent, 98% (UV)) were dissolved in 100 ml ethanol (Ethanol Absolut EP; Brenntag Schweizerhalle AG; Art.Nr. 82341-150) while stirring for 2 min at 24° C. to give a 0.1 molar solution. A PES flat sheet membrane (polyethersulfone, Ultrason® E 3010P from BASF SE, membrane thickness: 150-200 μm thickness, pore size 20-50 nm) for ultra filtration purposes was stored overnight in the ethanolic ninhydrin solution. The membrane was removed and dried for approx. 30 min at room temperature.

### Indicator Reaction

A drop of an albuminous sample, milk (3.5% fat) as protein source is applied to the open-pore (not glossy) side of the membrane. After 10 to 20 min at room temperature the sample has penetrated the membrane and the typical violet dye is formed both on the open-pore as well as on the dense (glossy) side of the membrane. A faster dye formation, within 5 to 10 min, is observed at elevated temperatures (30 to 40° C.).

### Example 2

### Membrane Treatment

1.78 g Ninhydrin (Ninhydrin; Fluka; Art.Nr. 72490-25G; ACS reagent, 98% (UV)) were dissolved in 100 ml ethanol (Ethanol Absolut EP; Brenntag Schweizerhalle AG; Art.Nr. 82341-150) while stirring for 2 min at 24° C. to give a 0.1 molar solution. A flat sheet membrane (NADIR® UP150 P, Microdyn-Nadir, polyethersulfone; backing material PE/PP, nominal MWCO of 150 kDa, membrane thickness: 210-250 μm) was stored overnight in the ethanolic ninhydrin solution. The membrane was removed and dried for approx. 30 min at room temperature.

The amount of ninhydrin adsorbed is summarized in table 1

### Indicator Reaction

Three drops of milk (3.5% fat) as protein source are applied side by side in parallel at a distance of at least 3 cm on the backing material of each membrane sample. The milk drops keep their shape after application on UP150 P. After 10 minutes at room temperature the milk has penetrated the membrane and visible color formation on the membrane occur, starting on the edge of the spread drops. After 20 minutes the color formation is clearly visible. The UP150 P is penetrated by the milk and migration through the membrane and visible color formation on both sides of the membrane occurs.

### Example 3

### Non-Inventive

### Membrane Treatment

1.78 g Ninhydrin (Ninhydrin; Fluka; Art.Nr. 72490-25G; ACS reagent, 98% (UV)) were dissolved in 100 ml ethanol (Ethanol Absolut EP; Brenntag Schweizerhalle AG; Art.Nr. 82341-150) while stirring for 2 min at 24° C. to give a 0.1 molar solution. A flat sheet membrane (NADIR® UV150 T, Microdyn-Nadir, polyvinylidene difluoride; backing material PET; nominal MWCO of 150 kDa, membrane thickness: 210-250 μm) was stored overnight in the ethanolic ninhydrin solution. The membrane was removed and dried for approx. 30 min at room temperature.

The amount of ninhydrin adsorbed is summarized in Table 1:

### Indicator Reaction

Three drops of milk (3.5% fat) as protein source are applied side by side in parallel at a distance of at least 3 cm on the backing material of each membrane sample. The milk drops spread quickly after application on UP150 T. The milk is very quickly absorbed by the membrane. After 10 minutes at room temperature the milk has penetrated the membrane and visible color formation on the membrane occur, starting on the edge of the spread drops. After 20 minutes the color formation is clearly visible. The UP150 T membrane penetrated by the milk but nearly no migration through the membrane takes place. Color formation is almost only on the side of the membrane visible, on which the milk is applied.

TABLE 1

| | | Weight | Weight | | Adsorbed |
|---|---|---|---|---|---|
| | Membrane | before | after | Weight | ninhydrin |
| | Area | treatment | treatment | difference | amount |
| Example | [cm²] | [g] | [g] | [g] | [g/m²] |
| 2 | 24.2 | 0.2549 | 0.26 | 0.0051 | 2.15 |
| 3* | 29.3 | 0.3894 | 0.4018 | 0.0124 | 4.1276 |

*Non-inventive

1. A fluid-absorbent article, comprising
(A) an upper liquid-pervious layer,
(B) a lower liquid-impervious layer and
(C) a fluid-absorbent core between (A) and (B), comprising 0 to 90% by weight fibrous material and 10 to 100% by weight water-absorbent polymer particles,
(D) and optionally an acquisition-distribution layer between (A) and (C), comprising 80 to 100% by weight fibrous material and 0 to 20% by weight water-absorbent polymer particles,
(E) and optionally at least one additional layer disposed immediately above and/or below (C)
and an indicator for proteins in bodily excretions, applied to a carrier material,
wherein the carrier material is any one of the upper liquid-pervious layer, the lower liquid-impervious layer, the fluid-absorbent core, the water-absorbent polymer particles and the additional layer.

2. A fluid-absorbent article, comprising
(A) an upper liquid-pervious layer,
(B) a lower liquid-impervious layer and
(C) a fluid-absorbent core between (A) and (B), comprising 0 to 90% by weight fibrous material and 10 to 100% by weight water-absorbent polymer particles,
(D) and optionally an acquisition-distribution layer between (A) and (C), comprising 80 to 100% by weight fibrous material and 0 to 20% by weight water-absorbent polymer particles,
(E) and optionally at least one additional layer disposed immediately above and/or below (C)
and an indicator for proteins in bodily excretions, applied to a carrier material,
wherein the carrier material is any one of a membrane or a layer fastened on the upper liquid-pervious layer, the lower liquid-impervious layer, the fluid-absorbent core or the additional layer.

3. A fluid-absorbent article according to claim 1, wherein the carrier material is hydrophilic.

4. A fluid-absorbent article according to claim 1, wherein the indicator is applied in distinct zones.

5. A fluid-absorbent article according to claim 1, wherein the indicator is applied according to a pattern.

6. A fluid-absorbent article according to claim 1, wherein the indicator comprises at least one indicator-substance which changes color as a result of contact with proteins.

7. A fluid-absorbent article according to claim 6, wherein the indicator-substance is ninhydrin.

8. A fluid-absorbent article according to claim 1, wherein the color change of the indicator substance is visible through the material of the liquid-impervious layer.

9. A fluid-absorbent article according to claim 8, wherein the liquid-impervious layer comprising a part having a color or transparency different from the rest of the layer.

10. A fluid-absorbent article according to claim 1, wherein the indicator is applied directly on the carrier material or indirectly by mixing with adhesives, varnishes, coatings, printing inks.

11. A fluid-absorbent article according to claim 2 wherein the membrane has a molecular weight cutoff (MWCO) of 10.000.000 Da.

12. A fluid-absorbent article according to claim 1, wherein the fluid-absorbent core comprises at least 60% by weight of fluid-absorbent polymer particles.

13. A fluid-absorbent article according to claim 1, wherein the fluid-absorbent core comprises less than 10% by weight of cellulose based fibers.

14. A fluid-absorbent article according to claim 1, wherein the fluid-absorbent polymer particles are placed in discrete regions of the fluid-absorbent core.

15. A fluid-absorbent article according to claim 1, wherein the fluid-absorbent core comprises at least 8 g of fluid-absorbent polymer particles.

16. A fluid-absorbent article according to claim 1, wherein the fluid-absorbent polymer particles have absorbency under high load of at least 18 g/g.

17. A fluid-absorbent article according to claim 1, wherein the fluid-absorbent polymer particles have a Centrifuge Retention Capacity at least of 25 g/g.

18. An indicator for proteins in bodily excretions, suitable for fluid-absorbent articles such as diapers, comprising at least one indicator-substance applied on a carrier material, which changes color as a result of contact with feces and or substances in urine indicating kidney or vascular diseases.

19. An indicator according to claim 18, whereas the carrier material is a membrane with a molecular weight cutoff (MWCO) of at least 100.000 Da.

20. An indicator according to claim 18 or 19, whereas the at least one indicator-substance is ninhydrin.
